(19) United States
(12) Patent Application Publication
(10) Pub. No.: US 2021/0349080 A1
(43) Pub. Date: Nov. 11, 2021

(54) MULTI-FACETED METHOD FOR DETECTING AND ANALYZING TARGET MOLECULE BY MOLECULAR APTAMER BEACON (MAB)

(71) Applicant: Shiqi LIAO, Lanzhou City (CN)

(72) Inventor: Shiqi LIAO, Lanzhou City (CN)

(21) Appl. No.: 17/148,630

(22) Filed: Jan. 14, 2021

(30) Foreign Application Priority Data
May 7, 2020 (CN) 202010378892.4

(51) Int. Cl. G01N 33/542; G01N 33/53; G01N 21/64

(52) U.S. Cl. CPC G01N 33/542; G01N 21/6458; G01N 33/5308

(57) ABSTRACT

A multi-faceted method for detecting and analyzing a target molecule by a molecular aptamer beacon is implemented by mixing an MAB and a test sample in a 1×binding buffer (BB) system with a carrier or in a suspension environment, incubating at 37-70° C. for 0.1-3 min where the MAB specifically binds to a target molecule in the test sample to form a multi-component complex and release a detection signal, detecting and analyzing with a detection instrument to achieve high-throughput and high-resolution imaging analysis and detection. When a molecular beacon binds to a target molecule, change of spatial structure of the molecular beacon causes an information label open, so that a variety of desired target molecules can be detected and identified qualitatively and quantitatively. Therefore, types of aptamer molecules and types of molecular beacons can be expanded and multiple detection methods can also be included.

### TECHNICAL FIELD

The present disclosure belongs to the field of molecular biological detection, and relates to a multi-faceted method for detecting and analyzing a target molecule by a molecular aptamer beacon (MAB).

### BACKGROUND

With continuous development in life sciences and chemistry, diagnostic technology in molecular biology develops rapidly while modern molecular biology and molecular genetics progress greatly, so that organisms are gradually known at a microscopic level. In recent years, many methods have been established for diagnosis at a molecular biology level, for example, restriction endonuclease analysis, nucleic acid molecular hybridization, and restriction fragment length polymorphism linkage analysis, achieving great progress. Molecular diagnostic technology reached a new height when Mullis, et al. in the Human Genetics Laboratory of Cetus, USA proposed the DNA in vitro amplification technology (polymerase chain reaction, PCR) in 1985 which developed rapidly afterwards, along with the DNA chip technology (DNA Chip) developed in the 1990s. However, detection technology in molecular biology still has shortcomings which need urgent improvement. For example, for the coronavirus disease 2019 (COVID-19), if there is high-throughput rapid screening and testing technology, a large number of people can be rapidly tested to find and isolate suspected patients, which will greatly slow down spread of the epidemic, save more people from suffering the disease and reduce great loss for countries. However, defects of the detection technology still present. For example, detection sensitivity of protein (pg level) and nucleic acid (molecular copy level) differs by more than 1,000 times, which greatly affects knowledge obtained from processes from genes to proteins to biological characterization. Other defects can be find in detection of spatial structure and diversity of protein, especially and more importantly, rapid and direct detection of molecules and biological samples. Breakthrough in rapid and direct detection of molecules will greatly promote development of biomedicine.

Molecular beacon is designed based on the principle of nucleic acid base pairing and the phenomenon of fluorescence resonance energy transfer (FRET) (FIG. 1). The FRET is a very interesting fluorescence phenomenon. When the fluorescence spectrum of a fluorescent molecule (also called a donor molecule) overlaps with the excitation spectrum of another fluorescent molecule (also called an acceptor molecule), excitation of the donor molecule can induce fluorescence of the acceptor molecule, and at the same time, fluorescence intensity of the donor molecule attenuates. This phenomenon is called FRET. Level of FRET closely relates to the spatial distance between the donor and acceptor molecules. FRET usually occurs at a distance of 7-10 mm, and as the distance increases, FRET decreases significantly by a factor of 10. Since the FRET is based on the principle of nucleic acid base pairing to bind target nucleic acid molecule, its application is limited to detection of nucleic acid molecules only (Prog. Biochem. Biophys. 1998; 25(6)).

Systematic evolution of ligands by exponential enrichment (SELEX) was initially used in 1990 by Tuerk, Ellington et al. to screen synthetic random oligonucleotide libraries to obtain high-affinity and strong-specific oligonucleotide ligands that bind to DNA polymerase of phage T4. The SELEX technology has developed into an important biotechnology for use in many fields such as basic research, drug screening, and toxicology research. Target molecules of aptamers are also expanding in type and number, including various biological macromolecules and especially small molecules, where certain progress has been made for small molecules.

A nucleic acid MAB is designed based on specific binding between an aptamer and a target molecule and FRET at a 5-8 bp neck of a stable structure (FIG. 2). Since the 5-8 bp neck of the structure cannot be opened at 37° C., corresponding methods are limited in development and application.

### SUMMARY

An objective of the present disclosure is to provide a multi-faceted method for detecting and analyzing a target molecule by an MAB, so as to detect and analyze the target molecule qualitatively and quantitatively in a simple, rapid, and accurate manner.

To this end, the following technical solutions are adopted in the present disclosure.

The method for detecting and analyzing a target molecule by an MAB of the present disclosure is implemented by mixing an MAB (see FIG. 3 for principle of detection with the MAB) and a test sample in a 1×BB (binding buffer) system with a carrier or in a suspension environment, incubating at 37-70° C. for 0.1-3 min where the MAB and a target molecule in the test sample are combined to form a multi-component complex and release a detection signal, detecting and analyzing with a detection instrument to achieve high-throughput and high-resolution imaging analysis and detection. In the present disclosure, the multi-component complex refers to multiple combinations of the MAB and the target molecule, that is, complexes formed by one or more MAB and one or more different epitope of the target molecule, or complexes formed by one or more MAB and one or more target molecule on a surface of a compound target substance.

The MAB is an artificially modified aptamer carrying a quencher which shows the same binding of an aptamer and a target molecule, and when the modified aptamer binds to a target molecule or a molecular structure thereof is changed, a detection signal can be released. The MAB has a structure including a head, a neck and a beacon. The head is an aptamer having a loop shape and a length of 10-60 bp or 6-40 amino acids. The head can specifically bind to the target molecule and can be polynucleotide or nucleic acid aptamer, polypeptide, peptide nucleic acid, oligosaccharide, antibody Fab, antibody mimic Fab, epitope, mimotope, cell receptor, ligand or biotin. The neck is a 3-8 bp complementary sequence which maintains a structure of a molecular beacon, and may be denatured and renatured when affected by temperature or external forces. The beacon part is responsible for molecular information emission. It can release corresponding signals when a molecular structure changes, for example, FRET.

The test sample is selected from the group consisting of biological samples, environmental samples, chemical samples, pharmaceutical samples, food samples, agricultural samples and veterinary samples.

The biological samples include whole blood, white blood cell, peripheral blood mononuclear cell, plasma, serum, sputum, exhaled breath, urine, semen, saliva, meningeal fluid, amniotic fluid, glandular fluid, lymph, nipple aspiration fluid, bronchial aspiration fluid, synovial fluid, joint aspiration fluid, cell, cell extract, stool, tissue, tissue extract, biopsy tissue, and cerebrospinal fluid.

The target molecule includes protein, peptide, carbohydrate, polysaccharide, glycoprotein, hormone, receptor, antigen, antibody, substrate, nucleic acid molecule, nucleic acid sequence, metabolite, target molecule analog, cofactor, inhibitor, drug, dye, nutrient, growth factor, cell, bacteria, chlamydia, virus, microcapsule, tissue and/or controlled substance, as well as any target molecule or substance containing target molecule that can specifically bind to the molecular beacon.

The carrier is selected from the group consisting of polymer bead, agarose bead, paramagnetic bead, glass bead, microtiter pore, cycloolefin copolymer substrate, membrane, plastic substrate, nylon, Langmuir-Bodgett membrane, nitrocellulose membrane, glass, silicon wafer chip, flow through chip, microbead, polytetrafluoroethylene substrate, polystyrene substrate, gallium arsenide substrate, gold substrate and silver substrate.

The detection signal includes light, electricity, magnetism, radiation, quantum dot, electrochemical signal and color developer.

When a solid carrier is used, the detection instrument may be a fully automatic laser scanning confocal microscope. When the suspension environment is used, the detection instrument may be a flow laser scanning confocal microscope.

The imaging analysis refers to computer analysis and processing based on detected strength of the signal released by the molecular beacon, for example, drawing a 3D map, analyzing signal strength, signal superpositioning, separating, and background eliminating.

The 1×BB solution may be prepared by adding 24.18 g of NaCl, 0.6 g of KCl, 8.7 g of Na₂HPO₄.12H₂O, 0.45 g of KH₂PO₄ and 0.6 g of MgCl₂.6H₂O into a conical flask, adding 800 ml of distilled water, stirring for dissolution, adjusting pH of the solution to 7.4 with HCl, adding distilled water to achieve a total volume of 1 L, and autoclaving for 20 min. The solution may be stored at room temperature.

In summary, the molecular beacon of the present disclosure is not limited to nucleic acid sequences or nucleic acid aptamers binding to target molecules, and not limited to cause FRET at the 5-8 bp neck. Rather, the present disclosure provides an artificially modified aptamer carrying a variety of information labels that can be opened based on specific binding of various aptamer molecules and target molecules. When a molecular beacon binds to a target molecule, change of spatial structure of the molecular beacon causes an information label open, so that various desired target molecules can be detected and identified qualitatively and quantitatively. Therefore, types of aptamer molecules and types of molecular beacons can be expanded and multiple detection methods can also be included as required.

### BRIEF DESCRIPTION OF DRAWINGS

FIG. 1 is a schematic diagram showing principle of nucleic acid detection by molecular beacon.

FIG. 2 is a diagram showing principle of MAB detection.

FIG. 3 is a diagram showing structure of MAB of the present disclosure and principle thereof. In FIG. 3, the term “Aptamer” refers to a nucleic acid sequence, nucleic acid aptamer, peptide nucleic acid, polypeptide, antibiotic, antibody Fab, epitope, receptor, ligand, biotin and any molecule that can bind to target molecule. The term “Neck” refers to a nucleic acid sequence, peptide nucleic acid sequence and amino acid and the like, as well as any controllable sequence. The term “Beacon” refers to light, electricity, magnetism, radiation, quantum dot, electrochemical signal and color developer and the like. The term “Target molecule” refers to a protein, peptide, carbohydrate, polysaccharide, glycoprotein, hormone, receptor, antigen, antibody, substrate, nucleic acid molecule, nucleic acid sequence, metabolite, target molecule analog, cofactor, inhibitor, drug, dye, nutrient, growth factor, cell, bacteria, chlamydia, virus, microcapsule, tissue and/or controlled substance, as well as any target molecule or substance containing target molecule that can specifically bind to the molecular beacon.

FIG. 4 is a diagram showing principle of detection of coronavirus in an exhaled breath by multiple fluorescent MABs of the present disclosure.

FIG. 5 is a diagram showing principle of detection by a peptide nucleic acid MAB of the present disclosure.

FIG. 6 is a diagram showing principle of detection of two epitopes of coronavirus S protein by multiple MABs of the present disclosure.

FIG. 7 is a diagram showing principle of capturing epitope 2 of S protein by aptamer and then detecting epitope 1 of S protein by multiplied MABs in the present disclosure.

FIG. 8 is a diagram showing principle of detection of a serum by multiple MABs of the present disclosure.

FIG. 9 is a diagram showing principle of detection of tumor pathological slice by the MAB of the present disclosure.

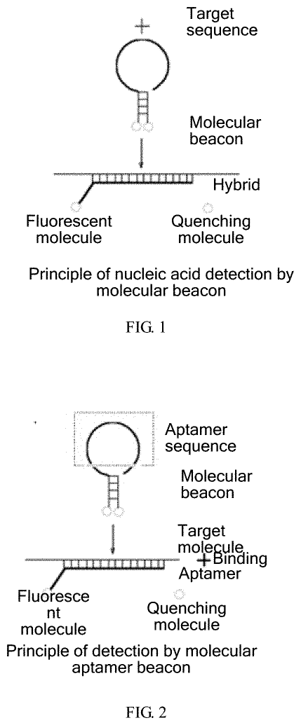

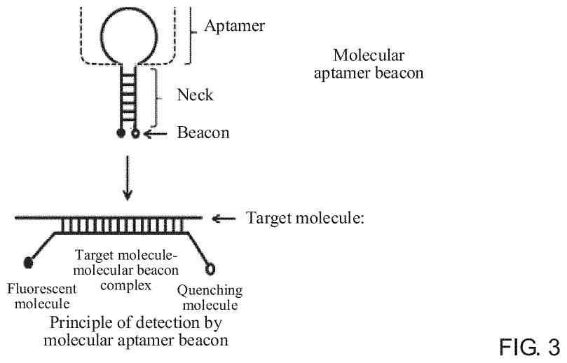

### DETAILED DESCRIPTION

### Example 1

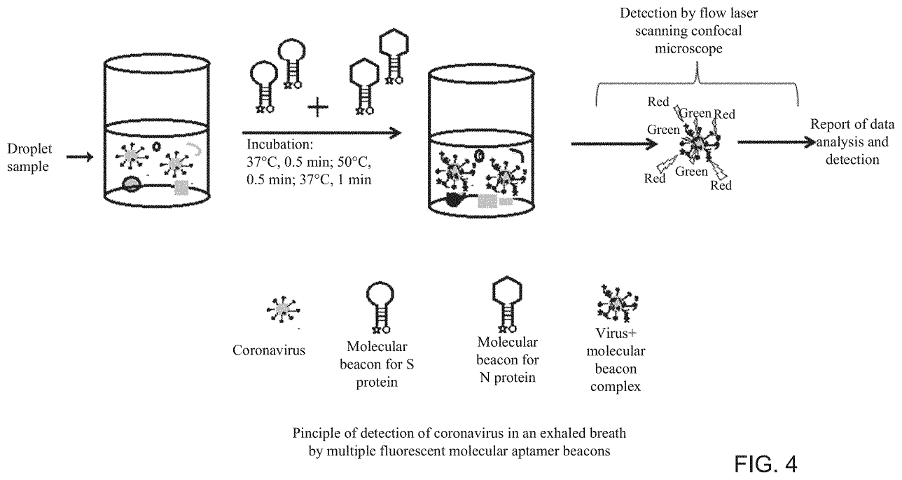

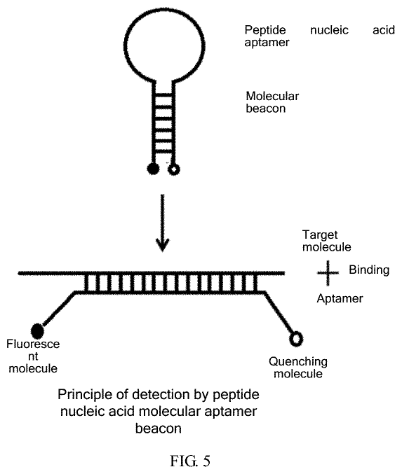

A multi-faceted method for detecting and analyzing coronavirus in an exhaled breath by multiple fluorescent MABs (FIG. 4) included the following steps.

Step (1): pathogen collection: an exhaled breath was collected by a quick freezing method. Breaths were exhaled into a quick freezer for 30 times. 1 mL of liquid was collected, and inactivated at 56° C. for 30 min to obtain pathogen containing exhaled breath liquid.

Step (2): formation of beacon complex: 10 pmol N protein nucleic acid MAB (with a fluorescent group FAM and a quenching group TAMER), 10 pmol of S protein nucleic acid MAB (fluorescent group CY5 and quenching group BYQ3) and 350 μL of 1×BB solution were added to the 1 mL pathogen containing exhaled breath liquid obtained in step (1), and incubated at 37° C. for 0.5 min, then 50° C. for 0.5 min and then 37° C. for 1 min to form a multi-component complex.

Step (3): detection and analysis: the formed multi-component complex was detected by a fully automatic flow laser scanning confocal microscope. Detected signals released by molecular beacons were analyzed and processed by computer. For example, qualitative and quantitative analysis of the test sample was carried out based on quantity of substance showing fluorescence in two colors and intensity. Two-color excitation light (green and red) superpositioned on a carrier with a diameter of 50-100 nm (diameter of virus) or more indicated a virus. The virus was quantified based on fluorescence intensity and quantity.

### Example 2

A multi-faceted method for detecting and analyzing Escherichia coli (E. coli) by multiple quantum dot MABs included the following steps.

Step (1): sample collection: 1.5 mL of test sample (for example, beverage or stool) was taken into a 5 mL centrifuge tube by a pipette, and centrifuged at 3,000 rpm for 10 min. A supernatant was taken to obtain a test sample liquid.

Step (2): formation of beacon complex: 10 pmol nucleic acid quantum dot MAB (with a fluorescent group CdTe and a quenching group AuNP) for E. coli lipopolysaccharide (LPS), 10 pmol nucleic acid quantum dot MAB for outer membrane protein (Omp), and 350 μL of 1×BB solution were added to 1 mL of the test sample liquid obtained in step (1) and incubated at 37° C. for 0.5 min, then 50° C. for 0.5 min and then 37° C. for 1 min to form a multi-component complex.

Step (3): detection and analysis: the formed multi-component complex was detected by a fully automatic flow laser scanning confocal microscope. Detected signals released by molecular beacons were analyzed and processed by computer. For example, qualitative and quantitative analysis of the test sample was carried out based on fluorescence intensity of a single or multiple E. coli substance(s) showing fluorescence in two colors.

### Example 3

A multi-faceted method for detecting and analyzing tumor cells in serum by a peptide nucleic acid MAB (FIG. 5) included the following steps.

Step (1): sample collection: 1.5 mL of blood was taken from vein, put into 5 mL centrifuge tube and centrifuged at 3,000 rpm for 10 min. A supernatant was discarded. A precipitate was washed with 1×BB and centrifuged at 3,000 rpm for 10 min. A supernatant was discarded to obtain a test sample.

Step (2): formation of beacon complex: 10 pmol nucleic acid MAB (with a fluorescent group FAM and a quenching group TAMER) for epithelial cell adhesion molecule (EpCAM) protein expressed on surfaces of circulating tumor cells (CTCs), and 1 mL of 1×BB solution were added to the test sample obtained in step (1), mixed and incubated at 37° C. for 0.5 min, then 50° C. for 0.5 min and then 37° C. for 1 min to form a multi-component complex.

Step (3): detection and analysis: the formed complex was detected by a fully automatic flow microscope. Detected signals released by the molecular beacon were analyzed and processed by computer, for example, drawing a 3D map, analyzing signal strength, signal superpositioning, separating, and eliminating. Therefore, qualitative and quantitative analysis of the test sample can be carried out, for example, based on number of cells that showed green fluorescence.

### Example 4

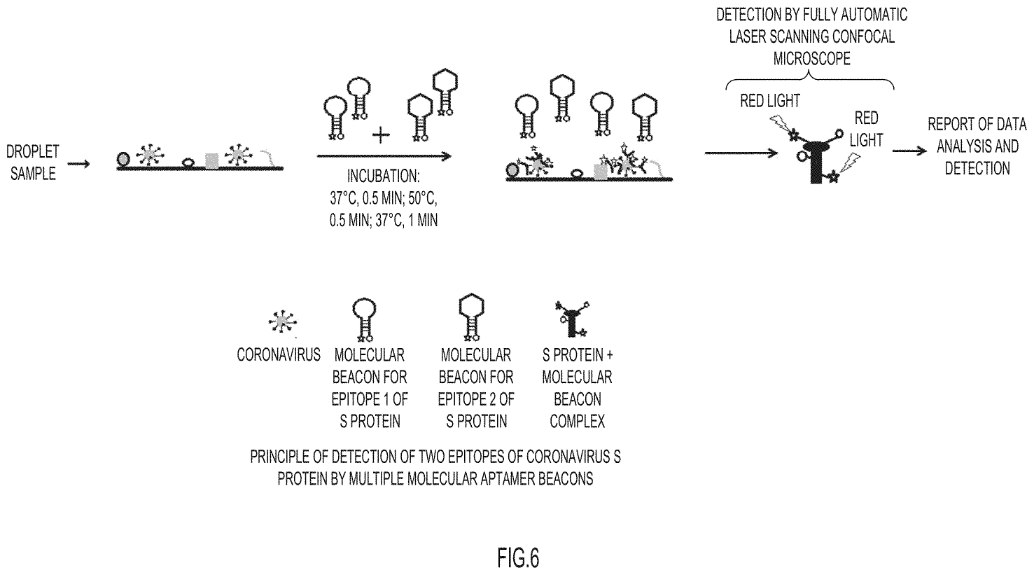

A multi-faceted method for detecting and analyzing two epitopes of coronavirus S protein by multiple MABs (see FIG. 6) included the following steps.

Step (1): sample collection: liquid in an exhaled breath was collected with a quick freezing method. Breaths were deeply exhaled 30 times into a quick freezer to collect 1 mL of liquid. The liquid was inactivated at 56° C. for 30 min, added with 2.5 ml of absolute ethanol, shaken, and centrifuged at 12,000 rpm for 30 min. A supernatant was discarded. A precipitate was washed twice with 75% ethanol, dissolved in 5 μL of 1×BB and dripped to a nitrocellulose filter membrane. 5 min later, cross linking was carried out under ultraviolet light for 6 s, and the membrane was put into a detection tube.

Step (2): formation of beacon complex: 10 pmol nucleic acid MAB (with a fluorescent group CY5 and a quenching group BYQ3) for epitope 1 of S protein, nucleic acid MAB (with a fluorescent group CY5 and a quenching group BYQ3) for epitope 2 of S protein and 100 μL of 1×BB solution were added to the detection tube in step (1), shaken gently and incubated at 37° C. for 0.5 min, then 50° C. for 0.5 min and then 37° C. for 1 min to form a multi-component complex.

Step (3): detection and analysis: detection was carried out with a front side of the nitrocellulose membrane (that is, the surface for dripping) facing a surface for excitation light of a fully automatic laser scanning confocal microscope. Detected signals released by molecular beacons were analyzed and processed by computer. For example, qualitative and quantitative analysis of the test sample was carried out by drawing a 3D map based on green fluorescence and red fluorescence of a scanned plane, superpositioning the two fluorescence signals, analyzing signal strength, separating and eliminating background.

### Example 5

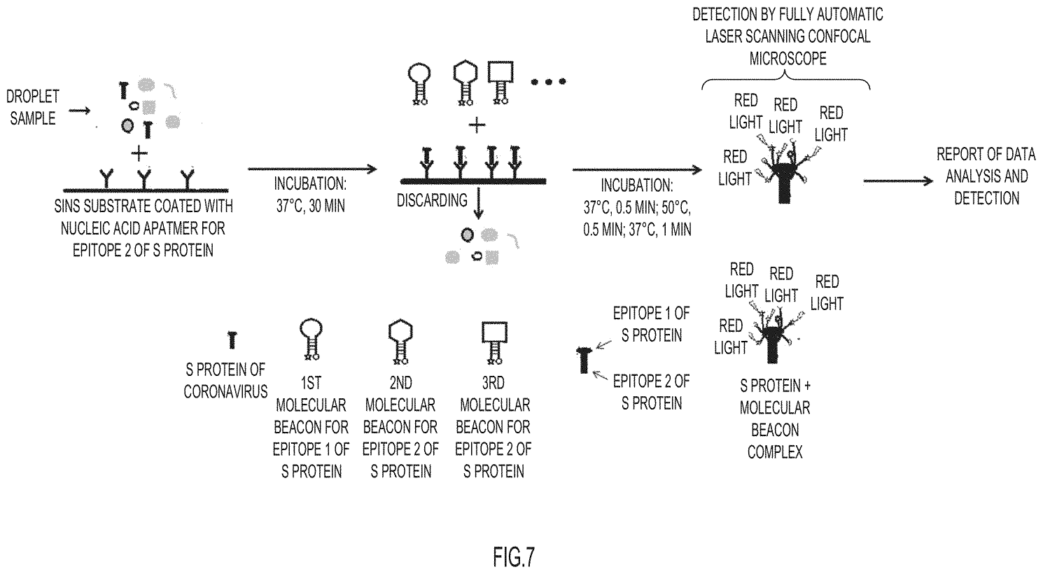

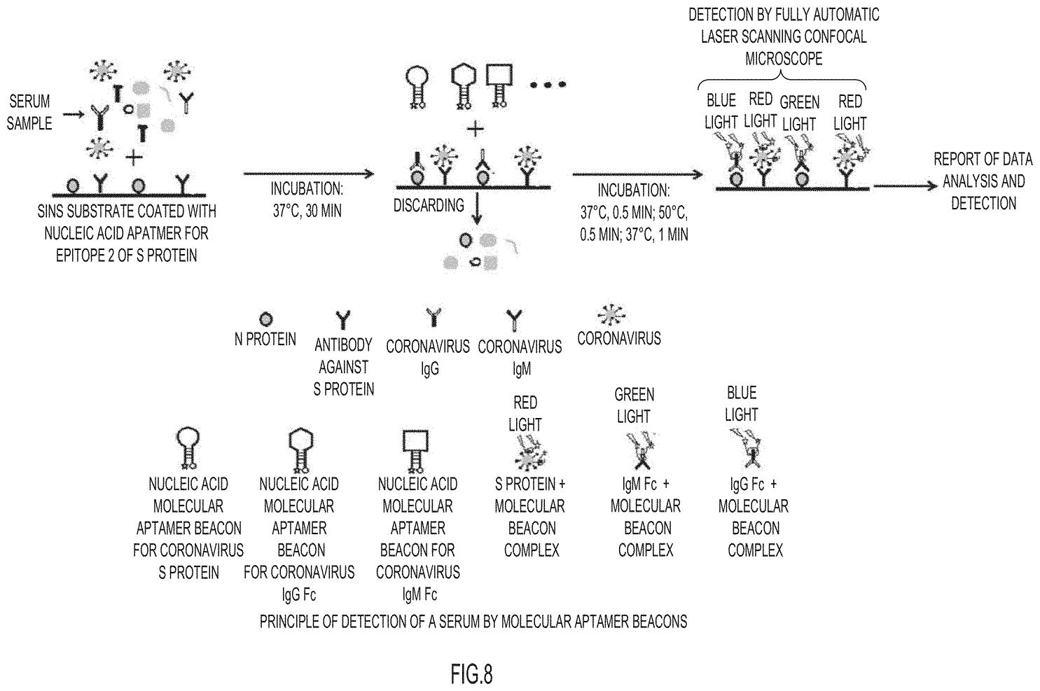

A multi-faceted method for capturing epitope 2 of S protein by aptamer and detecting epitope 1 of S protein by multiplied MABs (see FIG. 7) included the following steps:

Step (1): sample collection: liquid in an exhaled breath was collected with a quick freezing method. Breaths were deeply exhaled 30 times into a quick freezer to collect 1 mL of liquid. The liquid was inactivated at 56° C. for 30 min, added with 2.5 mL of absolute ethanol, shaken, and centrifuged at 12,000 rpm for 30 min. A supernatant was discarded. A precipitate was washed twice with 75% ethanol, dissolved in 5 μL of 1×BB and dripped to an SINS substrate coated with nucleic acid apatmer for epitope 2 of S protein (the SINS substrate connected to a streptavidin and a nucleic acid aptamer for biotinylated S protein epitope 2 thereof), shaken gently and incubated at 37° C. for 1 min.

Step (2): formation of beacon complex: 10 pmol multiple nucleic acid MABs (with a fluorescent group CY5 and a quenching group BYQ3) for epitope 1 of S protein (that is, multiple molecular beacon signals ( . . . (((epitope 1 of S protein-1st molecular beacon)-2nd molecular beacon)-3rd molecular beacon) . . . ) were formed from multiple aptamers obtained by multiple screening of epitope 1 of S protein in multiple libraries, and 100 μL of 1×BB solution were added to the SINS substrate in step (1), shaken gently and incubated at 37° C. for 0.5 min, then 50° C. for 0.5 min and then 37° C. for 1 min to form a multi-component complex.

Step (3): detection and analysis: detection was carried out with a front side of the SINS substrate (that is, the surface for dripping) facing a surface for excitation light of a fully automatic laser scanning confocal microscope. Detected signals released by molecular beacons were analyzed and processed by computer. For example, a 3D map was drawn based on green fluorescence of a scanned plane and processed, and the test sample was qualitatively and quantitatively analyzed based on signal strength.

### Example 6

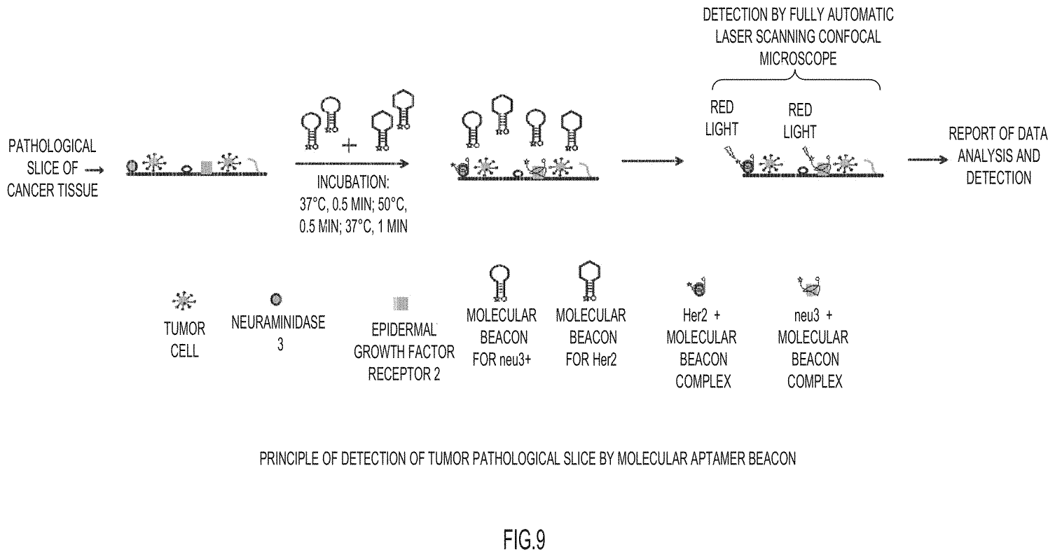

A multi-faceted method for detecting S protein-IgG-IgM protein in serum by multiple MABs (see FIG. 8) included the following steps.

Step (1): sample collection: 1.5 mL of blood was taken from vein, put in a 5 mL centrifuge tube, and centrifuged at 3,000 rpm for 10 min. A supernatant was taken, inactivated at 56° C. for 30 min, added with 2.5 mL of absolute ethanol, shaken, and centrifuged at 12,000 rpm for 30 min. A supernatant was discarded. A precipitate was washed twice with 75% absolute solution, dissolved with 5 μL of 1×BB and dripped to an SINS substrate coated with or to different areas of an antibody against coronavirus S protein and N protein, shaken gently, and incubated at 37° C. for 1 min.

Step (2): formation of beacon complex: 10 pmol nucleic acid MAB (with a fluorescent group CY5 and a quenching group BYQ3) for epitope 1 of S protein, 10 pmol nucleic acid MAB (with a fluorescent group ATT0425 and a quenching group BYQ2) for IgG Fc, 10 pmol nucleic acid MAB (with a fluorescent group FAM and a quenching group TAMER) for IgM Fc, and 100 μL of 1 λBB solution were added to the SINS substrate in step (1), shaken gently and incubated at 37° C. for 0.5 min, then 50° C. for 0.5 min and then 37° C. for 1 min to form a multi-component complex.

Step (3): detection and analysis: detection was carried out with a front side of the SINS substrate (that is, the surface for dripping) facing a surface for excitation light of a fully automatic laser scanning confocal microscope. Detected signals released by molecular beacons were analyzed and processed by computer. For example, a 3D map was drawn based on green, blue and red fluorescence of a scanned plane. Then the three-color (or regional) fluorescence signals were processed. Then, the test sample was qualitatively and quantitatively analyzed based on signal strength. Or qualitative and quantitative analysis of the test sample was carried out by detecting the three-color fluorescence by a fully automatic flow laser scanning confocal microscope directly based on combination of the S protein-IgG-IgM protein in a liquid with molecular beacons.

### Example 7

A multi-faceted method for detecting and analyzing tumor pathological slice by MABs (see FIG. 9) included the following steps.

Step (1): sample collection: a paraffin pathological slice of invasive ductal breast cancer was prepared based on a paraffin pathological slice preparation process adopted by a pathology department.

Step (2): formation of beacon complex: 10 pmol neu3 nucleic acid MAB (with a fluorescent group CY5 and a quenching group BYQ3), 10 pmol Her2 nucleic acid MAB (with a fluorescent group FAM and a quenching group TAMER) and 100 μL of 1×BB solution were added to the pathological slice of step (1), shaken gently, and incubated at 37° C. for 0.5 min, then 50° C. for 0.5 min, and then 37° C. for 1 min to form a multi-component complex.

Step (3): detection and analysis: detection was carried out with a front side of the slice (that is, the surface for dripping) facing a surface for excitation light of a fully automatic laser scanning confocal microscope. Detected signals released by molecular beacons were analyzed and processed by computer. For example, a 3D map was drawn based on green and red fluorescence of a scanned plane. The two fluorescence signals were superpositioned, and signal strength was analyzed. Separation was carried out and background was eliminated, so that the test sample was qualitatively and quantitatively analyzed.

What is claimed:

1. A multi-faceted method for detecting and analyzing a target molecule by a molecular aptamer beacon (MAB), comprising:
mixing an MAB and a test sample in a 1×binding buffer (BB) system with a carrier or in a suspension environment;
incubating at 37-70° C. for 0.1-3 min, wherein the MAB specifically binds to a target molecule in the test sample to form a multi-component complex and release a detection signal; and
detecting and analyzing with a detection instrument to achieve high-throughput and high-resolution imaging analysis and detection.

2. The method of claim 1, wherein:
the MAB is an artificially modified aptamer having a neck-loop structure, and comprises a head, a neck and a beacon, wherein the head is an aptamer having a loop shape and a length of 10-60 bp or 6-40 amino acids, is configured to specifically bind to the target molecule and can be polynucleotide or nucleic acid aptamer, polypeptide, peptide nucleic acid, oligosaccharide, antibody Fab, antibody mimic Fab, epitope, mimotope, cell receptor, ligand or biotin;
the neck is a 3-8 bp complementary sequence; and
the beacon is responsible for molecular information emission, and is configured to release a corresponding signal when a molecular structure changes.

3. The method of claim 1, wherein the test sample is selected from the group consisting of a biological sample, an environmental sample, a chemical sample, a pharmaceutical sample, a food sample, an agricultural sample, and a veterinary sample.

4. The method of claim 3, wherein the test sample is a biological sample, and the biological sample comprises whole blood, white blood cell, peripheral blood mononuclear cell, plasma, serum, sputum, exhaled breath, urine, semen, saliva, meningeal fluid, amniotic fluid, glandular fluid, lymph, nipple aspiration fluid, bronchial aspiration fluid, synovial fluid, joint aspiration fluid, cell, cell extract, stool, tissue, tissue extract, biopsy tissue, or cerebrospinal fluid.

5. The method of claim 1, wherein the target molecule comprises protein, peptide, carbohydrate, polysaccharide, glycoprotein, hormone, receptor, antigen, antibody, substrate, nucleic acid molecule, nucleic acid sequence, metabolite, target molecule analog, cofactor, inhibitor, drug, dye, nutrient, growth factor, cell, bacteria, chlamydia, virus, microcapsule, tissue and/or controlled substance, or any target molecule or substance containing target molecule that specifically binds to the molecular beacon.

6. The method of claim 1, wherein the carrier is selected from the group consisting of polymer bead, agarose bead, paramagnetic bead, glass bead, microtiter pore, cycloolefin copolymer substrate, membrane, plastic substrate, nylon, Langmuir-Bodgett membrane, nitrocellulose membrane, glass, silicon wafer chip, flow through chip, microbead, polytetrafluoroethylene substrate, polystyrene substrate, gallium arsenide substrate, gold substrate. and silver substrate.

7. The method of claim 1, wherein the detection signal comprises light, electricity, magnetism, radiation, quantum dot, electrochemical signal and color developer.

8. The method of claim 1, wherein, when a solid carrier is used, the detection instrument is a fully automatic laser scanning confocal microscope, and when the suspension environment is used, the detection instrument is a flow laser scanning confocal microscope.
